(19) United States
(12) Patent Application Publication
(10) Pub. No.: US 2008/0003291 A1
(43) Pub. Date: Jan. 3, 2008

(54) Antibiotic and deodorant material and a preparation method thereof

(75) Inventors: Hsien-Yi Chiu, Gangshan Town (TW); Yu-Cheng Liu, Kaohsiung City (TW)

Correspondence Address: Robert F. Gazdzinski, Esq.;Gazdzinski & Associates, Suite 375, 11440 West Bernardo Court, San Diego, CA, 92127, US

(73) Assignee: TAIWAN TEXTILE RESEARCH INSTITUTE, Taipei Hsien (TW)

(21) Appl. No.: 11/649,942

(22) Filed: Jan. 4, 2007

(30) Foreign Application Priority Data
Jun. 30, 2006 (TW) 95123937

(51) Int. Cl. A61K 9/14; A61K 33/38

(52) U.S. Cl. 424/489; 424/618

(57) ABSTRACT

An antibiotic and deodorant material and a preparation method thereof are provided. At first, a mesoporous molecular sieve, a grafting agent and a solvent are reacted under a thermal flux system. The grafting agent has a structure of chemical formula I or II, wherein R₁ and R₂ are alkyl groups having 1 to 20 carbon atoms. The grafting agent can modify surfaces of the mesoporous molecular sieve. Then, the surfaces of the mesoporous molecular sieve are modified by silver ions. Finally, the mesoporous molecular sieve is reduced by a reducing agent to form an antibiotic and deodorant material.

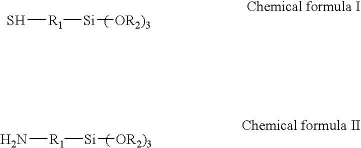

### RELATED APPLICATIONS

The present application is based on, and claims priority from, Taiwan Application Serial Number 95123937, filed Jun. 30, 2006, the disclosure of which is hereby incorporated by reference herein in its entirety.

### BACKGROUND

1. Field of Invention

The present invention relates to a material and a preparation method thereof. More particularly, the present invention relates to an antibiotic and deodorant material and a preparation method thereof.

2. Description of Related Art

In 1992, American Mobil Company successfully developed mesoporous molecular sieves with high regularity in pore size by using quaternary ammonium salt as a template to react with. aluminosilicate. From that time, more and more people have started to study properties of mesoporous molecular sieves. Because mesoporous molecular sieves have many advantages such as large surface area, high pore size regularity, high thermal stability and adjustable pore size, many uses of mesoporous molecular sieves have been discovered. Because mesoporous molecular sieves have large surface area and high regular pores, mesoporous molecular sieves are very suitable to be a deodorant material.

Silver has good antibiotic property but its price is high. However, silver can be nano-sized to reduce usage amount and production cost. Nano-sized siliver particles with large surface areas and good dispersion property provide better antibiotic property.

Traditional preparation method of an antibiotic and deodorant material is to directly adhere silver nanoparticles to mesoporous molecular sieves. Because silver nanoparticles are so small in size, silver nanoparticles easily depart from mesoporous molecular sieve and enter into human bodies from skin to threaten health once if physical adhesion force of silver nanoparticles to mesoporous molecular sieves is insufficient,.

### SUMMARY

It is therefore an aspect of the present invention to provide an antibiotic and deodorant material and a preparation method thereof. The antibiotic and deodorant material has excellent antibiotic and deodorant properties. Moreover, the antibiotic and deodorant material prevents silver nanopariticles from entering into human body.

In accordance with the foregoing and other aspects of the present invention, a preparation method of an antibiotic and deodorant material is provided. Firstly, mesoporous molecular sieves react with a grafting agent having a structure of chemical formula I or II in a solvent under a thermal flux system. In the chemical formula I or II, R₁ and R₂ are alkyl groups having 1 to 20 carbon atoms. Si—OR₂ functional groups having the grafting agent react with hydroxyl groups on surfaces of the mesoporous molecular sieves by a condensation reaction to form Si—O—Si bonds. Then, the mesoporous molecular sieves are washed with a silver salt solution to let S atoms or N atoms of the grafting agent coordinate to silver ions in the silver salt solution to form coordinate bonds of Ag—S or Ag—N. Finally, the mesoporous molecular sieves react with a reducing agent to reduce the silver ions thereon to form the antibiotic and deodorant material.

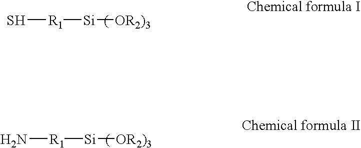

In accordance with the foregoing and other aspects of the present invention, a preparation method of an antibiotic and deodorant material is provided. Firstly, mesoporous molecular sieves react with a grafting agent having a structure of chemical formula I or II in a solvent under a thermal flux system. In the chemical formula I or II, R₁ and R₂ are alkyl groups having 1 to 20 carbon atoms. The grafting agent modifies surfaces of the mesoporous molecular sieves. Then, the surfaces of the mesoporous molecular sieves are modified with silver ions. Finally, the mesoporous molecular sieves react with a reducing agent to reduce the silver ions thereon to form the antibiotic and deodorant material.

In accordance with the foregoing and other aspects of the present invention, an antibiotic and deodorant material is provided. The antibiotic and deodorant material is mesoporous molecular sieves. Functional groups having chemical formula III or IV are on surfaces of the mesoporous molecular sieves. In the chemical formula III or IV, R₃ is an alkyl group having 1 to 20 carbon atoms.

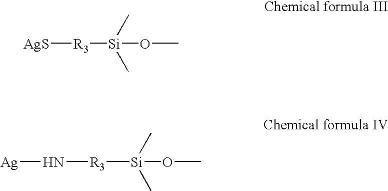

In conclusion, in the antibiotic and deodorant material, silver atoms are chemically bonded on the mesoporous molecular sieves. Because chemical bonding force is stronger than physically adhesion force, silver atoms hardly depart from surfaces of the mesoporous molecular sieves to enter into human body. Therefore, this antibiotic and deodorant material have no threaten to human's health. Moreover, the mesoporous molecular sieves have high stability and large surface area. When silver atoms are bonded on the mesoporous molecular sieves, not only the dispersion property of silver atoms but also antibiotic property thereof can be increased.

### DETAILED DESCRIPTION

In the preparation method of an antibiotic and deodorant material according to an embodiment of the invention, mesoporous molecular sieves react with a grafting agent in a solvent under a thermal flux system. The grafting agent has a structure of chemical formula I or II. In the chemical formula I or II, R₁ and R₂ are alkyl groups having 1 to 20 carbon atoms. The grafting agent can modify surfaces of the mesoporous molecular sieves. Si—OR₂ functional groups having the grafting agent can react with hydroxyl groups on the surfaces of the mesoporous molecular sieves by a condensation reaction to form Si—O—Si bonds. Then, silver ions are used to modify the surfaces of the mesoporous molecular sieves. The mesoporous molecular sieves are washed with a silver salt solution to let S atoms or N atoms of the grafting agent coordinate to silver ions in the silver salt solution to form coordinate bonds of Ag—S or Ag—N. Finally, the mesoporous molecular sieves react with a reducing agent to reduce the silver ions thereon to form the antibiotic and deodorant material.

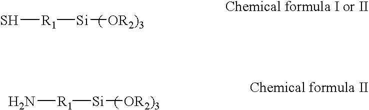

In a embodiment, the material of the mesoporous molecular sieve can be silicon oxide. The pore shape of the mesoporous molecular sieve can be hexagonal. The solvent can be alcohol such as methanol, ethanol, propyl alcohol or butyl alcohol. The silver salt solution can be a silver nitrate solution, a silver sulfate solution, a silver fluoride solution, a silver acetate solution, a silver oxide solution or a silver fluoborate solution. The reducing agent can be a sodium borohydride solution, a formaldehyde solution, a sodium bisulphate solution, an iodine bromide solution, a sodium amalgam solution, a sodium carbonate anhydrous solution, a sodium carbonate anhydrous solution, a sodium biphthalate solution, an oxalic acid solution, a glucose solution or a sodium thiosulfate solution.

The antibiotic and deodorant material is a modified mesoporous molecular sieves. The backbone material and pore shape are not affected by the surface modifying reactions described above. Functional groups of a chemical formula III or IV are on surfaces of the mesoporous molecular sieves. In the chemical formula III or IV, R₃ is an alkyl group of 1 to 20 carbon atoms,

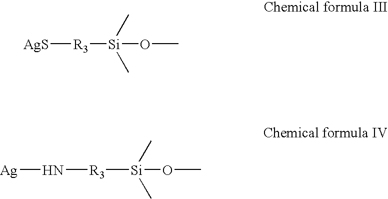

In a working example, the mesoporous molecular sieves were prepared. The preparation method of the mesoporous molecular sieves can refer to Microporous and Mesoporous Material 80, (2005), 221-226. The material of the mesoporous molecular sieves was silicon oxide. The pore shape of the mesoporous molecular sieves was hexagonal. The particle size of the mesoporous molecular sieves was determined by a multi-wavelength laser particle size analyzer and an electron microscope. The average particle size of the mesoporous molecular sieves was about 1000 nm. The structure of the mesoporous molecular sieves was also determined by an X-ray powder diffractometer. The pore diameter of the mesoporous molecular sieves was about 5-6 nm and the pore spacing thereof was about 11 nm. The surface area of the mesoporous molecular sieves was measured by nitrogen adsorption-desorption method. The surface area of the mesoporous molecular sieves was about 300 to 1500 m²/g. Moreover, the mesoporous molecular sieves had excellent gas adsoption ability.

At first, 50 g of the mesoporous molecular sieves reacted with 50 g of a grafting agent in ethanol under a thermal flux system of 80° C. The grafting agent used here was MPTS (3-Mercaptopropyl trimethoxysilane). After the reaction, the mesoporous molecular sieves were washed and purified. Then, the mesoporous molecular sieves were well mixed and reacted with a silver salt solution by ultra sonic vibration. The silver salt solution was prepared by mixing 750 g of water and 1.2 g of silver nitrate. During the reaction, silver ions of the silver salt solution modified surfaces of the mesoporous molecular sieves. After the reaction, the mesoporous molecular sieves were washed and purified again. Then, the mesoporous molecular sieves were well mixed and reacted with a reducing agent by ultra sonic vibration. The reducing agent was prepared by mixing 250 g of water and 0.6 g of sodium borohydride. During the reaction, the reducing agent reduced silver ions on the surfaces of the mesoporous molecular sieves to form an antibiotic and deodorant material.

The silver amount in the antibiotic and deodorant material was measured by inductively coupled plasma-atomic emission spectrometry (ICP-AES). The measured silver amount in the antibiotic and deodorant material was 8000 ppm. The atom composition of the antibiotic and deodorant material was also be determined by energy dispersive x-ray spectrometer (EDX) to observe Ag—S bond. The ratio of S atom to Ag atom was about 3:10 to 1:1.

In the traditional biotic and deodorant material, silver nanoparticles are physically adhered to mesoporous molecular sieves. Because physical adhesion force is weak, silver nanoparticles easily depart from surfaces of mesoporous molecular sieve and enter into human body to threaten human health. In the antibiotic and deodorant material of the invention, silver atoms are chemically bonded to mesoporous molecular sieves. Because chemical bonding force is stronger than physical adhesion force, silver atom will not depart from mesoporous molecular sieves and entering into human body.

Following are separately deodorant test and antibiotic test.

### Deodorant Test

Table 1 is a comparison table of deodorant test result of the antibiotic and deodorant material of the invention, activate carbon and bamboo charcoal. 0.14 g of sample was put into a container full of nitrogen. Nitrogen concentrations of the container before putting the sample and after putting the sample for 30 minutes were measured separately. Then, deodorant rate could be obtained by the decrease in nitrogen concentration. From the comparison table, the antibiotic and deodorant material of the invention had better deodorant ability than the other two deodorant materials, active carbon and bamboo charcoal. The antibiotic and deodorant material of the invention had the best deodorant ability among these three materials, the bamboo charcoal is the second, and the active carbon is the last.

| | Ammonia concentration | | |
|---|---|---|---|
| | (ppm) | | |
| Sample | 0 minute | 30 minute | Deodorant rate (%) |
| an antibiotic and | 780 | 200 | 75 |
| deodorant material | | | |
| of the invention | | | |
| activate carbon | 660 | 400 | 39 |
| bamboo charcoal | 520 | 230 | 56 |

### Antibiotic Test.

The antibiotic and deodorant material can be added into water-soluble polyurethane and then be coated on a polyester fabric to form an antibiotic and deodorant textile.

Table 2 is a comparison table of antibiotic test result of the antibiotic and deodorant textile of the invention. The biotic standard of table 2 was based on the biotic standard of Japanese Association for the Functional Evaluation of Textiles (JAFET). The test method was JIS L1902-1998. When bacteriostasis value is large than 2.2, test samples have bacteriostasis effect. When bactericidal value is large than 0, test samples have bactericidal effect. In the test, the coated substrate was polyester fabric. The coated amount was 38 g/m². The weight percentages of the antibiotic and deodorant material in water-soluble polyurethane were 0%, 1% and 5%. In the table, the uncoated polyurethane had no antibiotic ability. After coated with water-soluble polyurethane of the antibiotic and deodorant material, the antibiotic ability of the polyester fabric was apparently improved. With the increase in the amount of the antibiotic and deodorant material coated on the polyester fabric, the antibiotic ability of the polyester fabric apparently increased.

| | | Weight percentages | | |
|---|---|---|---|---|
| | | of the antibiotic | | |
| | | and deodorant | | |
| | | material in | | |
| | | water-soluble | | |
| | | polyurethane (wt %) | | |
| Test item | | 0% | 1% | 5% |
| Staphylococcus | Bacteriostasis | 2.1 | 5.0 | >5.9 |
| aureus | value | | | |
| | Bactericidal | <0 | 2.1 | >3.0 |
| | value | | | |

Accordingly, the present invention has the following advantages.

(1) The antibiotic and deodorant material has excellent antibotic and deodorant property. The mesoporous molecular sieves of the antibiotic and deodorant material not only have high absorbing gas ability but also increase silver dispersion property and antibiotic ability.

(2) The antibiotic and deodorant material of the invention prevents silver nanoparticles from entering into human body.

The embodiments of the present invention described above should not be regarded as limitations to the present invention. It will be apparent to those skilled in the art that various modifications and variations can be made to the present invention without departing from the scope or spirit of the invention. The scope of the present invention is as defined in the appended claims.

What is claimed is:

1. A preparation method of an antibiotic and deodorant material, comprising:
reacting mesoporous molecular sieves with a grafting agent having a structure of chemical formula I or II in a solvent under a thermal flux system, 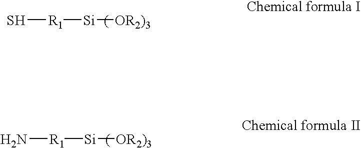
wherein R₁ and R₂ are alkyl groups having 1 to 20 carbon atoms, and the Si—OR₂ functional groups having the grafting agent are reacted with hydroxyl groups on surfaces of the mesoporous molecular sieves by a condensation reaction to form Si—O—Si bonds;
washing the mesoporous molecular sieves with a silver salt solution to let S atoms or N atoms of the grafting agent coordinate to silver ions in the silver salt solution to form coordinate bonds of Ag—S or Ag—N; and
using a reducing agent to reduce the silver ions on the mesoporous molecular sieves to form the antibiotic and deodorant material.

2. The method of claim 1, wherein the pore shape of the mesoporous molecular sieve is hexagonal.

3. The method of claim 1, wherein the material of the mesoporous molecular sieve is silicon oxide.

4. The method of claim 1, wherein the silver salt solution is a silver nitrate solution, a silver sulfate solution, a silver fluoride solution, a silver acetate solution, a silver oxide solution or a silver fluoborate solution.

5. The method of claim 1, wherein the reducing agent is a sodium borohydride solution, a formaldehyde solution, a sodium bisulphate solution, an iodine bromide solution, a sodium amalgam solution, a sodium carbonate anhydrous solution, a sodium carbonate anhydrous solution, a sodium biphthalate solution, an oxalic acid solution, a glucose solution or a sodium thiosulfate solution.

6. The method of claim 1, wherein the solvent is alcohol.

7. The method of claim 6, wherein the alcohol is methanol, ethanol, propyl alcohol or butyl alcohol,

8. An antibiotic and deodorant material, comprising mesoporous molecular sieves having functional groups of chemical formula III or IV on surfaces thereof, wherein R₃ is an alkyl group having 1 to 20 carbon atoms,

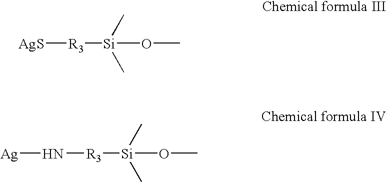

9. The antibiotic and deodorant material of claim 8, wherein the material of the mesoporous molecular sieve is silicon oxide.

10. The antibiotic and deodorant material of claim 8, wherein the pore shape of the mesoporous molecular sieve is hexagonal.

11. A preparation method of an antibiotic and deodorant material, comprising:
reacting mesoporous molecular sieves with a grafting agent having a structure of chemical formula I or II in a solvent in a thermal reflux system, 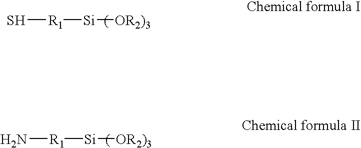
wherein R₁ and R₂ are alkyl groups having 1 to 20 carbon atoms, and the grafting agent modifies surfaces of the mesoporous molecular sieves;
modifying the surfaces of the mesoporous molecular sieves with silver ions; and
using a reducing agent to reduce the silver ions on the mesoporous molecular sieves to form the antibiotic and deodorant material.

12. The method of claim 11, wherein the material of the mesoporous molecular sieve is silicon oxide.

13. The method of claim 11, wherein the pore shape of the mesoporous molecular sieve is hexagonal.

14. The method of claim 11, wherein the reducing agent is a sodium borohydride solution, a formaldehyde solution, a sodium bisulphate solution, an iodine bromide solution, a sodium amalgam solution, a sodium carbonate anhydrous solution, a sodium carbonate anhydrous solution, a sodium biphthalate solution, an oxalic acid solution, a glucose solution or a sodium thiosulfate solution.

15. The method of claim 11, wherein the solvent is alcohol.

16. The method of claim 15, wherein the alcohol is methanol, ethanol, propyl alcohol or butyl alcohol,
